(19) United States
(12) Patent Application Publication
(10) Pub. No.: US 2016/0331580 A1
(43) Pub. Date: Nov. 17, 2016

(54) MASSAGING DEVICE HAVING FLUID CONTAINER

(71) Applicant: Paul CHEN, Vancouver (CA)

(72) Inventor: Paul CHEN, Vancouver (CA)

(21) Appl. No.: 14/708,408

(22) Filed: May 11, 2015

(51) Int. Cl. A61F 7/00; A61H 1/00

(52) U.S. Cl. CPC A61F 7/00; A61H 1/008; A61F 2007/0088; A61F 2007/0092; A61F 2007/0091

(57) ABSTRACT

A massaging device includes a housing having a chamber and a mouth communicating with the chamber of the housing for filling a fluid into the chamber of the housing, and a cover engaged with the mouth of the housing for enclosing the chamber of the housing and for retaining the fluid within the chamber of the housing and for preventing the fluid from flowing out of the chamber of the housing. The housing includes a number of peripheral protrusions for forming one or more peripheral recesses. The cover includes one or more depressions for forming a knob. The housing includes a casing extended into the housing, and the casing includes an inner end portion engaged with the cover.

### BACKGROUND OF THE INVENTION

1. Field of the Invention

The present invention relates to a massaging device, and more particularly to a massaging device including a fluid containing structure or configuration for containing the heated fluid or hot water and for providing a heat to comfortably heat and massage the user.

2. Description of the Prior Art

Typical massaging devices comprise a relatively harder or stronger inner housing, and a relatively softer or resilient outer layer for selectively contacting or engaging with the users and for massaging purposes.

For example, U.S. Pat. No. 4,807,603 to Yasui discloses one of the typical massaging devices also comprising a relatively harder or stronger inner housing having a cylindrical shape or contour or structure, and a relatively softer or resilient outer layer for selectively contacting or engaging with the users and for massaging purposes.

However, the typical massaging devices include a solid structure or configuration having no space or compartment to receive and contain the heated fluid or hot water such that the typical massaging devices may not provide a heat to comfortably heat and massage the user.

U.S. Pat. No. 8,888,808 to Park discloses another typical massaging device comprising a planar base member having a number of projections or protrusions extended outwardly therefrom for frictionally contacting or engaging with the users and for massaging purposes.

However, similarly, the typical massaging devices also include a solid structure or configuration having no space or compartment to receive and contain the heated fluid or hot water such that the typical massaging devices may not provide a heat to comfortably heat and massage the user.

The present invention has arisen to mitigate and/or obviate the afore-described disadvantages of the conventional massaging devices.

### SUMMARY OF THE INVENTION

The primary objective of the present invention is to provide a massaging device including a fluid containing structure or configuration for containing the heated fluid or hot water and for providing a heat to comfortably heat and massage the user.

In accordance with one aspect of the invention, there is provided a massaging device comprising a housing including a chamber formed therein, and including a mouth formed in the housing and communicating with the chamber of the housing for filling a fluid, such as a heated fluid or hot water, into the chamber of the housing, and a cover engaged with the mouth of the housing for enclosing the chamber of the housing and for retaining the fluid within the chamber of the housing and for preventing the fluid from flowing out of the chamber of the housing. The heat provided from the heated fluid or hot water may suitably warm up the muscles and nerves of the user and may increase or facilitate the massaging effect to the user.

The housing includes a number of peripheral protrusions extended outwardly therefrom for forming at least one peripheral recess between the peripheral protrusions respectively and for massaging purposes and/or for increasing the friction between the user and the housing.

The housing includes an inner thread formed in the mouth, and the cover includes an outer thread for engaging with the inner thread of the housing and for attaching the cover to the mouth of the housing, or for selectively detaching or disengaging the cover from the mouth of the housing.

The cover includes at least one depression formed therein for forming or defining a knob and for being grasped or operated with the user, and arranged for allowing the user to thread or to unthread the cover from the mouth of the housing. The housing includes a casing extended into the chamber of the housing, and the casing preferably includes an inner end portion engaged with the cover for anchoring or positioning the cover in place.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a perspective view of a massaging device in accordance with the present invention;

FIG. 2 is an exploded view of the massaging device as seen from one direction; and

FIG. 3 is a cross sectional view of the massaging device, taken along lines 3-3 of FIG. 1, illustrating the operation of the massaging device.

### DETAILED DESCRIPTION OF THE PREFERRED EMBODIMENT

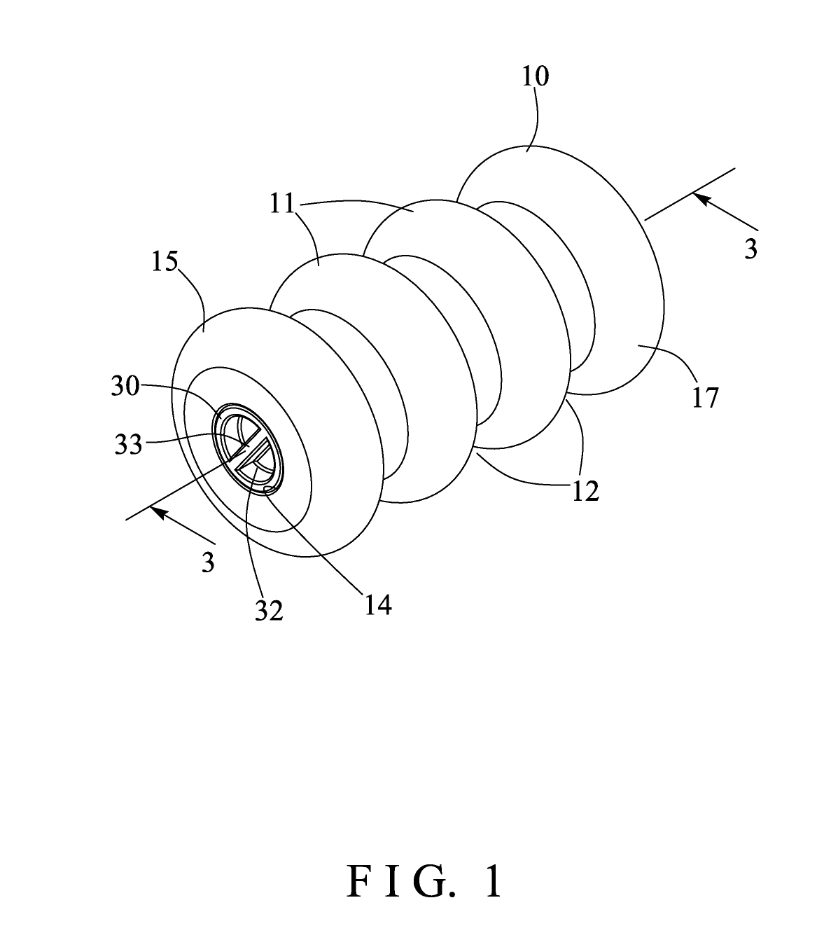

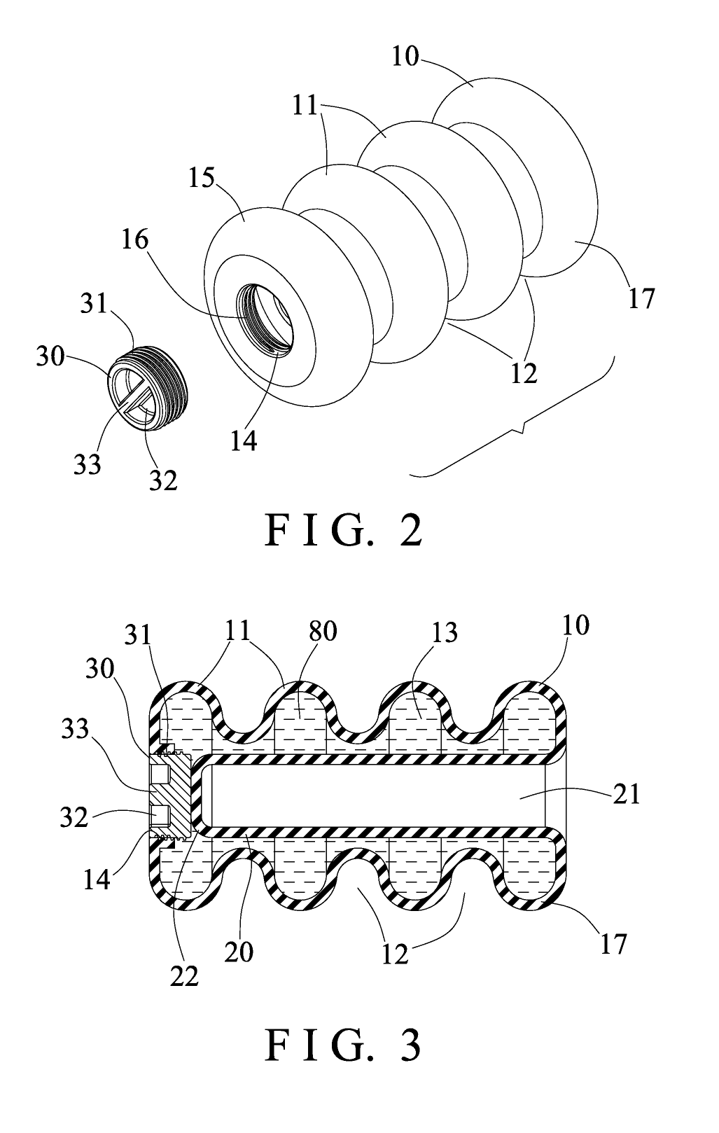

Referring to the drawings, and initially to FIGS. 1 and 2, a massaging device in accordance with the present invention comprises an outer receptacle or housing 10 made of a relatively softer or resilient material, such as plastic, synthetic materials, rubber or the like for softly and comfortably contacting or engaging with the users and for massaging purposes, the housing 10 preferably includes a substantially cylindrical shape or contour having one or more (such as four) annular or circular and peripheral protrusions 11 extended radially and outwardly therefrom, for forming or defining one or more (such as four) annular or circular and peripheral recesses 12 therein and located between the peripheral protrusions 11 respectively.

As shown in FIG. 3, the housing 10 includes an inner compartment or bore or chamber 13 formed therein for receiving or containing a water or fluid 80 therein, such as a heated fluid or hot water 80 therein, and includes an opening or mouth 14 formed in one of two end portions 15, 17 thereof and communicating with the chamber 13 of the housing 10 for filling or discharging the fluid 80 into or out of the chamber 13 of the housing 10, and includes an inner thread 16 formed in the mouth 14 for threading or engaging with a lid or cover 30. For example, the cover 30 includes an outer thread 31 formed or provided thereon for threading or engaging with the inner thread 16 of the housing 10 and for detachably or changeably attaching the cover 30 to the mouth 14 of the housing 10.

The cover 30 includes one or more (such as two) notches or depressions 32 formed therein for forming or defining a relatively protruded or projected knob 33 thereon and for being grasped or operated with the user, and arranged for allowing the user to thread or to unthread the cover 30 from the mouth 14 of the housing 10. The cover 30 may thus be selectively and detachably or changeably attached to the mouth 14 of the housing 10 for blocking or enclosing the chamber 13 of the housing 10 and for solidly and stably retaining the fluid 80 within the chamber 13 of the housing 10, and for preventing the fluid 80 from flowing out of the chamber 13 of the housing 10.

As also shown in FIG. 3, the housing 10 includes an inner tubular member or casing 20 extended from the other end portion 17 of the housing 10 and into the chamber 13 of the housing 10, and the casing 20 includes a chamber or compartment 21 formed therein and spaced or separated from the chamber 13 of the housing 10 by the casing 20, and communicating with the outer environment for suitably reducing the volume of the fluid receiving chamber 13 of the housing 10, or for suitably reducing the volume or quantity of the fluid 80 that is required to be filled into the chamber 13 of the housing 10. It is preferable, but not necessary that the casing 20 includes an inner end portion 22 contacted or engaged with the cover 30 for further anchoring or positioning the cover 30 in place to the housing 10.

In operation, as shown in FIG. 2, the water or fluid 80, such as a heated fluid or hot water 80 may be filled or introduced into the chamber 13 of the housing 10 from the mouth 14 of the housing 10, and the cover 30 may then be engaged with the mouth 14 of the housing 10 for blocking or enclosing the chamber 13 of the housing 10 and for solidly and stably retaining the fluid 80 within the chamber 13 of the housing 10, and for preventing the fluid 80 from flowing out of the chamber 13 of the housing 10. The heated fluid or hot water 80 received and contained in the chamber 13 of the housing 10 may provide a heat to suitably and comfortably heat and massage the user.

It is to be noted that the typical massaging devices have no space or compartment to receive and contain the heated fluid or hot water such that the typical massaging devices may not provide a heat to comfortably heat and massage the user. The heat provided from the heated fluid or hot water 80 may suitably warm up the muscles and nerves of the user and may further increase the massaging effect to the user.

Accordingly, the massaging device in accordance with the present invention includes a fluid containing structure or configuration for containing the heated fluid or hot water and for providing a heat to comfortably heat or warm up the muscles of the user and for massaging the user.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A massaging device comprising:
a housing including a chamber formed therein, and including a mouth formed in said housing and communicating with said chamber of said housing for filling a fluid into said chamber of said housing, and
a cover engaged with said mouth of said housing for enclosing said chamber of said housing and for retaining the fluid within said chamber of said housing and for preventing the fluid from flowing out of said chamber of said housing.

2. The massaging device as claimed in claim 1, wherein said housing includes a plurality of peripheral protrusions extended outwardly therefrom for forming at least one peripheral recess between said peripheral protrusions respectively.

3. The massaging device as claimed in claim 1, wherein said housing includes an inner thread formed in said mouth, and said cover includes an outer thread for engaging with said inner thread of said housing and for attaching said cover to said mouth of said housing.

4. The massaging device as claimed in claim 1, wherein said cover includes at least one depression formed therein for defining a knob.

5. The massaging device as claimed in claim 1, wherein said housing includes a casing extended into said chamber of said housing.

6. The massaging device as claimed in claim 5, wherein said casing includes an inner end portion engaged with said cover.
